(19) United States
(12) Patent Application Publication
(10) Pub. No.: US 2021/0076924 A1
(43) Pub. Date: Mar. 18, 2021

(54) ENDOSCOPE AND DISTAL END COVER

(71) Applicant: OLYMPUS CORPORATION, Tokyo (JP)

(72) Inventor: Koji YAMAYA, Tokyo (JP)

(73) Assignee: OLYMPUS CORPORATION, Tokyo (JP)

(21) Appl. No.: 17/106,662

(22) Filed: Nov. 30, 2020

Related U.S. Application Data
(63) Continuation of application No. PCT/JP2019/006567, filed on Feb. 21, 2019.

(30) Foreign Application Priority Data
Jun. 1, 2018 (JP) 2018-105956

(51) Int. Cl. A61B 1/12; A61B 1/015; A61B 1/00

(52) U.S. Cl. CPC A61B 1/126; A61B 1/00137; A61B 1/015

(57) ABSTRACT

An endoscope includes a distal end member including a fluid passage, a first distal end cover made of an elastically deformable material, a second distal end cover made of a material harder than that of the first distal end cover, and a nozzle. The nozzle includes a nozzle channel portion communicating with the fluid passage and an ejection port. The endoscope further includes a first nozzle configuring portion provided to the first distal end cover, configuring at least part of the nozzle, and configuring at least part of a channel bending portion having a bent shape in the nozzle channel portion. The first nozzle configuring portion is covered by the second distal end cover in a mounted state.

### CROSS REFERENCE TO RELATED APPLICATION

This application is a continuation application of PCT/JP2019/006567 filed on Feb. 21, 2019 and claims benefit of Japanese Application No. 2018-105956 filed in Japan on Jun. 1, 2018, the entire contents of which are incorporated herein by this reference.

### BACKGROUND OF THE INVENTION

### 1. Field of the Invention

The present invention relates to an endoscope provided with a nozzle for cleaning at a distal end portion of an insertion portion and a distal end cover.

### 2. Description of the Related Art

Endoscopes have been widely used in recent years in a medical field and an industrial field. An endoscope used in the medical field includes an elongated insertion portion that is inserted into a human body, and is widely used for such as observation of an organ, treatment with a treatment instrument, surgery under endoscopic observation.

A distal end portion of the insertion portion is provided with an observation window positioned at a distalmost end of an image pickup optical system and a nozzle for cleaning a surface of the observation window. When such as blood, body fluid, or dirt adhere to the surface of the observation window, cleaning water is ejected from the nozzle to remove the adhering matter and then air is ejected from the nozzle to remove water droplets remaining on the surface of the observation window.

Incidentally, such as the blood, the body fluid, or the dirt may not only adhere to the surface of the observation window, but also intrude into a fluid conduit communicating with the nozzle. The fluid conduit therefore needs to be reliably cleaned by such as brushing with a cleaning brush after use of the endoscope.

Japanese Patent Application Laid-Open Publication No. 5-317235 describes an endoscope in which an observation window cleaning nozzle (hereinafter simply referred to as nozzle) for directing an air/water feeding passage to the observation window is formed on a distal end cover made of elastic rubber. The endoscope allows a conduit cleaning brush to be inserted into the air/water feeding tube fixed to a distal end portion body, by detaching the distal end cover from the distal end portion body.

### SUMMARY OF THE INVENTION

An endoscope of one aspect of the present invention includes: a distal end member provided at a distal end portion of an insertion portion configured to be inserted in a subject and including a fluid passage for allowing a fluid to flow; a first distal end cover made of an elastically deformable material and detachably attached to the distal end member; a second distal end cover made of a material harder than the material of the first distal end cover and attached to the distal end member; a nozzle including a nozzle channel portion communicating with the fluid passage in a mounted state in which the first distal end cover and the second distal end cover are attached to the distal end member, and an ejection port positioned at an end of the nozzle channel portion and configured to eject the fluid to an outside; and a first nozzle configuring portion provided to the first distal end cover, configuring at least part of the nozzle, and configuring at least part of a bending portion having a bent shape in the nozzle channel portion. The first nozzle configuring portion is covered by the second distal end cover in the mounted state.

A distal end cover of one aspect of the present invention is a distal end cover detachably attached to a distal end member including a fluid passage for allowing a fluid to flow through at a distal end portion of an insertion portion of an endoscope. The distal end cover includes: a first cover made of an elastically deformable material and detachably attached to the distal end member; a second cover made of a material harder than the material of the first cover and attached to the distal end member; a nozzle including a nozzle channel portion communicating with the fluid passage in a mounted state in which the first cover and the second cover are attached to the distal end member, and an ejection port positioned at an end of the nozzle channel portion and configured to eject the fluid to an outside; and a first nozzle configuring portion provided to the first cover, configuring at least part of the nozzle, and configuring at least part of a bending portion having a bent shape in the nozzle channel portion. The first nozzle configuring portion is covered by the second cover in the mounted state.

### BRIEF DESCRIPTION OF THE DRAWINGS

FIG. 1 is an explanatory diagram of a configuration of an endoscope according to a first embodiment of the present invention.

FIG. 2 is a perspective view of a distal end portion according to the first embodiment of the present invention.

FIG. 3 is a perspective view of a disassembled distal end portion illustrated in FIG. 2.

FIG. 4 is a plan view of the disassembled distal end portion illustrated in FIG. 2.

FIG. 5 is a side view of a distal end member according to the first embodiment of the present invention.

FIG. 6 is a plan view of a state in which a first distal end cover is joined to a second distal end cover according to the first embodiment of the present invention.

FIG. 7 is a cross-sectional view of a midway state of attaching first and second distal end covers to the distal end member according to the first embodiment of the present invention.

FIG. 8 is a cross-sectional view of a normal mounted state according to the first embodiment of the present invention.

FIG. 9 is a cross-sectional view of a first abnormal mounted state example according to the first embodiment of the present invention.

FIG. 10 is a cross-sectional view of a second abnormal mounted state example according to the first embodiment of the present invention.

FIG. 11 is a perspective view of a detaching jig according to the first embodiment of the present invention.

FIG. 12 is a perspective view of a state in which a fragile portion of the second distal end cover is broken according to the first embodiment of the present invention.

FIG. 13 is a perspective view of a distal end portion according to a second embodiment of the present invention.

FIG. 14 is a perspective view of the disassembled distal end portion illustrated in FIG. 13.

FIG. 15 is a plan view of a distal end member according to the second embodiment of the present invention.

FIG. 16 is a side view of the distal end member according to the second embodiment of the present invention.

FIG. 17 is a cross-sectional view of a midway state of attaching first and second distal end covers to the distal end member according to the second embodiment of the present invention.

FIG. 18 is a cross-sectional view of a mounted state according to the second embodiment of the present invention.

FIG. 19 is a perspective view of a distal end portion according to a third embodiment of the present invention.

FIG. 20 is a perspective view of a disassembled distal end portion illustrated in FIG. 19.

FIG. 21 is a cross-sectional view of a midway state of attaching first and second distal end covers to a distal end member according to the third embodiment of the present invention.

FIG. 22 is a cross-sectional view of a mounted state according to the third embodiment of the present invention.

FIG. 23 is a perspective view of a distal end portion of an insertion portion of a forward viewing endoscope.

FIG. 24 is a perspective view of the distal end member of the distal end portion illustrated in FIG. 23, the first distal end cover, and the second distal end cover that are disassembled.

FIG. 25 is a cross-sectional view of a state before the first and second distal end covers illustrated in FIG. 24 are attached to the distal end member.

FIG. 26 is a cross-sectional view of a state in which the first and second distal end covers illustrated in FIG. 24 are attached to the distal end member.

FIG. 27 is a perspective view of a detaching jig used to detach the first and second distal end covers illustrated in FIG. 24 from the distal end member.

FIG. 28 is a perspective view illustrating a state in which a fragile portion of the second distal end cover illustrated in FIG. 24 is broken.

### DETAILED DESCRIPTION OF THE PREFERRED EMBODIMENTS

Hereinafter, embodiments of the present invention will be described with reference to the accompanying drawings.

### First Embodiment

### (Configuration of Endoscope)

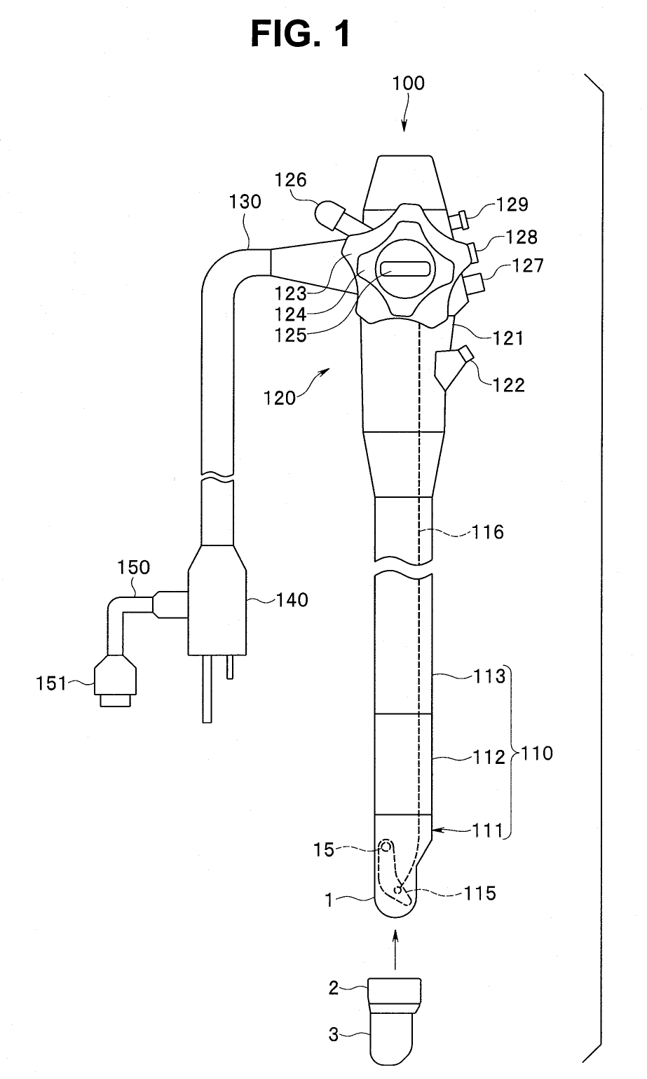

A schematic configuration of an endoscope according to a first embodiment of the present invention will be described first. FIG. 1 is an explanatory diagram of a configuration of an endoscope 100 according to the present embodiment. The endoscope 100 is configured as a side viewing endoscope or an oblique viewing endoscope. The endoscope 100 includes an insertion portion 110 inserted into a subject, an operation portion 120 connected to a proximal end of the insertion portion 110, a universal cord 130 extended from the operation portion 120, and a connector 140 provided at the distal end of the universal cord 130.

The insertion portion 110 has an elongated shape, and includes a distal end portion 111 that is positioned at the distal end of the insertion portion 110, a bending portion 112 that is bendable, and a flexible tube portion 113 that has flexibility. The distal end portion 111, the bending portion 112, and the flexible tube portion 113 are connected to each other in this order from the distal end side of the insertion portion 110.

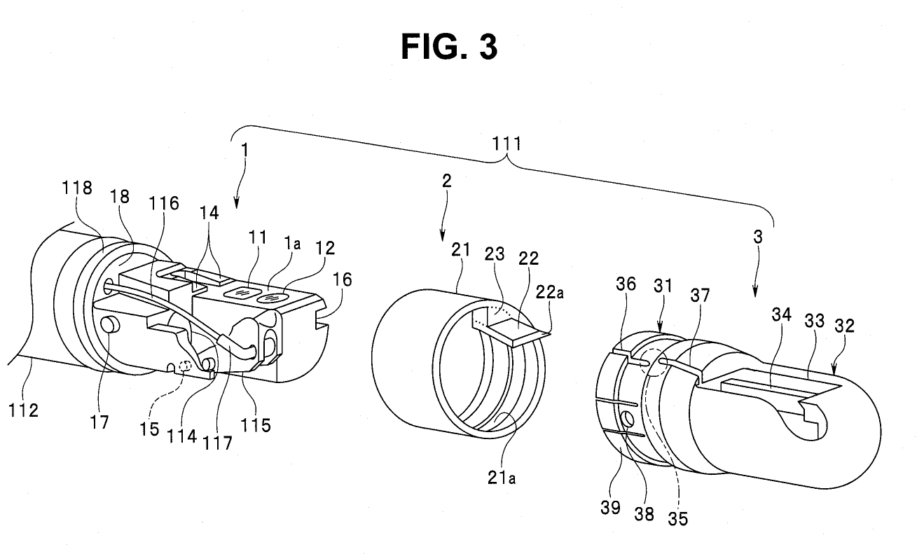

The distal end portion 111 includes an image pickup optical system and an illumination optical system that are optical members for observation, an image pickup apparatus, a treatment instrument outlet port 114 that is an opening portion of a treatment instrument channel provided in the insertion portion 110, and a raising base (forceps elevator) 115 that raises the treatment instrument. Note that only the raising base 115 is illustrated in FIG. 1. The treatment instrument outlet port 114 is illustrated such as in FIG. 3 described later. The treatment instrument outlet port 114 also serves as a suction port. The image pickup apparatus includes an image pickup device that picks up an image of an object.

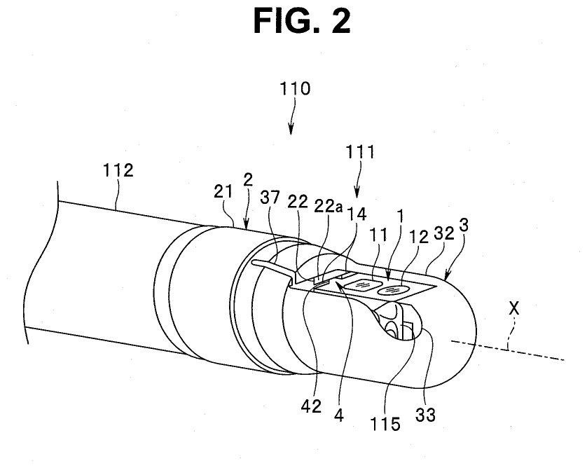

The endoscope 100 further includes a distal end member 1 constituting the distal end portion 111, a first distal end cover 2, a second distal end cover 3, and a nozzle 4. The distal end member 1 is provided at the distal end portion 111, and is a main portion of the distal end portion 111. The first distal end cover 2 and the second distal end cover 3 are attached to the distal end member 1. The nozzle 4 is illustrated such as in FIG. 2 described later. The distal end member 1, the first distal end cover 2, the second distal end cover 3, and the nozzle 4 will be described later in detail.

The operation portion 120 includes a grasping portion 121. The grasping portion 121 is connected to the proximal end of the flexible tube portion 113. The grasping portion 121 is provided with a treatment instrument insertion port 122 communicating with the treatment instrument channel.

The operation portion 120 is provided with two bending operation knobs 123 and 124, and a fixing lever 125. The bending portion 112 of the insertion portion 110 is configured to be bent in four directions, for example, up, down, right, and left, by a turning operation of the bending operation knobs 123 and 124. The fixing lever 125 fixes the bending operation knobs 123 and 124 in a desired turning position.

The operation portion 120 is further provided with a raising base operation lever 126. A raising base operation wire (hereinafter simply referred to as wire) 116 connected to the raising base 115 and the raising base operation lever 126 is provided in the insertion portion 110 and the operation portion 120. The raising base operation lever 126 manipulates the pulling or loosening of the wire 116. The pulling or loosening of the wire 116 by the raising base operation lever 126 causes the raising base 115 to rise or fall.

The operation portion 120 is further provided with an air/water feeding button 127, a suction button 128, and a cleaning tube attachment cap 129. The air/water feeding button 127 is a button to control an air feeding function and a water feeding function of the endoscope 100. The suction button 128 is a button to control a suction function of the endoscope 100.

The endoscope 100 is connected to external devices such as an air/liquid feeding device, a light source device, and a processor, which are not illustrated, via the connector 140. The connector 140 is provided with a signal transmission cable 150 extending from a side portion of the connector 140. An electrical connector 151 connected to the processor is provided at the distal end of the signal transmission cable 150.

### (Configuration of Distal End Portion)

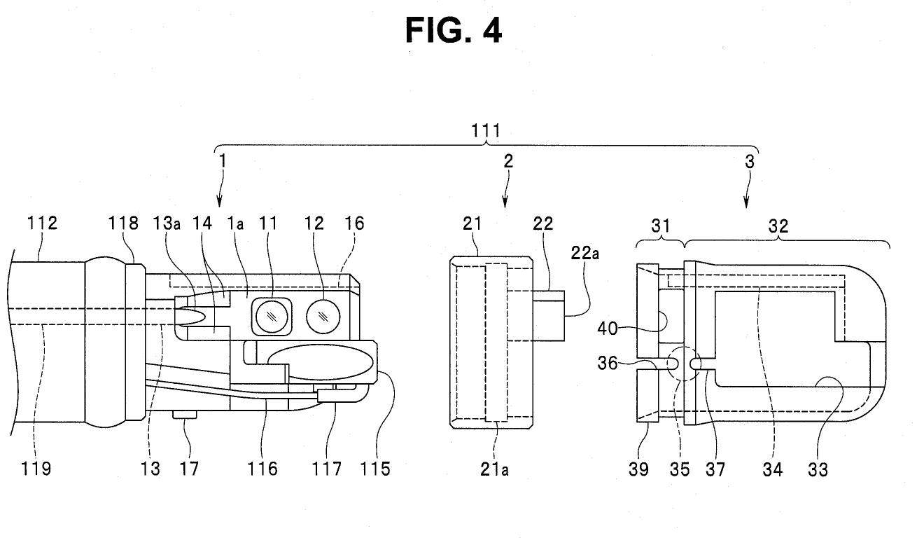

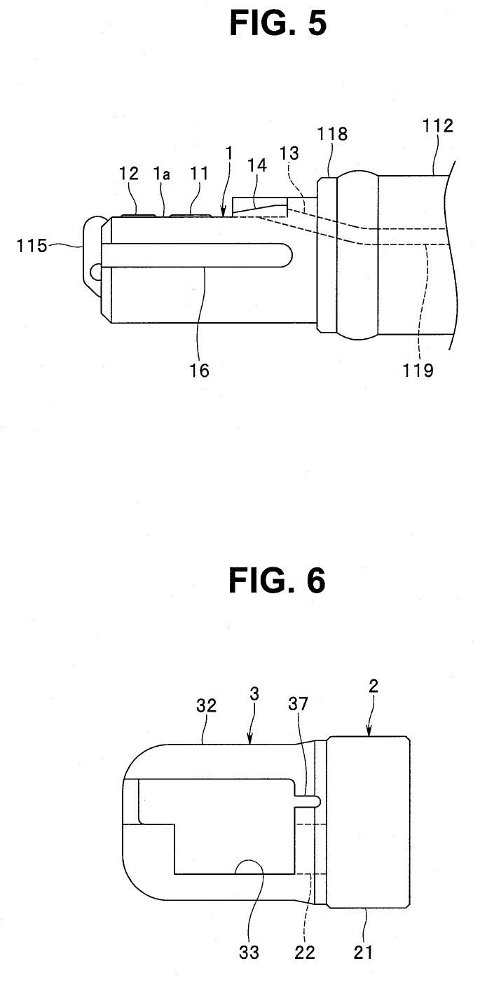

The configuration of the distal end portion 111 will be described in detail with reference to FIG. 2 to FIG. 5 next. FIG. 2 is a perspective view of the distal end portion 111. FIG. 3 is a perspective view of the disassembled distal end portion 111. FIG. 4 is a plan view of the disassembled distal end portion 111. FIG. 5 is a side view of the distal end member 1. An insertion axis X of the insertion portion 110 (central axis of insertion portion 110) is indicated by a dashed-and-dotted line in FIG. 2. In FIG. 3 and FIG. 4, depicted are the distal end member 1, the first distal end cover 2, and the second distal end cover 3 separated from each other in a direction parallel to the insertion axis X illustrated in FIG. 2.

The distal end member 1 is made of a metal material such as stainless steel. The first distal end cover 2 is made of an elastically deformable material, and is detachably attached to the distal end member 1. The second distal end cover 3 is made of a material harder than that of the first distal end cover 2, and is attached to the distal end member 1.

A rubber material such as silicone rubber, for example, is used as a material for forming the first distal end cover 2. A resin material such as polysulfone resin, for example, is used as a material for forming the second distal end cover 3. Both of the first distal end cover 2 and the second distal end cover 3 are preferably formed of an insulation material that satisfies the above-described requirements relating to the material to prevent a high frequency current from leaking to the outside of the insertion portion 110, for a case a high frequency treatment instrument such as a high frequency electrical scalpel is used as a treatment instrument.

Hereinafter, the configuration of the distal end member 1 will be described. The distal end member 1 has a side surface 1a parallel to the insertion axis X of the insertion portion 110 as illustrated in FIG. 2 to FIG. 5. An observation window 11 positioned at a distalmost end of the image pickup optical system and an illumination window 12 positioned at a distalmost end of the illumination optical system are provided on the side surface 1a of the distal end member 1. The observation window 11 and the illumination window 12 are arranged side by side along the insertion axis X of the insertion portion 110.

The nozzle 4 of the present embodiment serves to clean the surfaces of the observation window 11 and the illumination window 12, and is configured to be able to blow a predetermined fluid to the observation window 11 and the illumination window 12. The predetermined fluid is cleaning water and air, for example. The distal end member 1 further includes a fluid passage 13 for allowing the predetermined fluid to flow through, and a second nozzle configuring portion 14 configuring part of the nozzle 4 as illustrated in FIG. 2 to FIG. 5. The fluid passage 13 has an opening 13a positioned at an end of the fluid passage 13, and communicates with an air/water feeding conduit 119 provided inside the insertion portion 110. The second nozzle configuring portion 14 includes two portions protruding from the side surface 1a of the distal end member 1 and arranged to sandwich the opening 13a of the fluid passage 13.

The distal end member 1 further includes a rotation shaft 15 as illustrated in FIG. 3. The raising base 115 is rotatably attached to the rotation shaft 15. The distal end member 1 is provided with the treatment instrument outlet port 114 through which the treatment instrument is led out. The treatment instrument is raised by the raising base 115. The treatment instrument outlet port 114 and the raising base 115 are arranged side by side along the insertion axis X of the insertion portion 110. The raising base 115 is made of a metal material such as stainless steel, for example.

A wire terminal member 117 is provided at the distal end of the wire 116 as illustrated in FIG. 3 and FIG. 4. The wire 116 is connected to the raising base 115 via the wire terminal member 117. The pulling or loosening of the wire 116 causes the raising base 115 to rise and fall in a direction around the axis of the rotation shaft 15. Note that the raising base 115 and the wire terminal member 117 may be configured such that the wire terminal member 117 is detachably connected to the raising base 115.

The distal end member 1 further includes a restricting recess 16 and a locking pin 17 as illustrated in FIG. 3 to FIG. 5. The restricting recess 16 and the locking pin 17 are provided in a portion except for the side surface 1a in an outer peripheral portion of the distal end member 1. The restricting recess 16 is a groove portion formed in the outer peripheral portion of the distal end member 1 along the insertion axis X of the insertion portion 110. The locking pin 17 is a protrusion protruding from the outer peripheral portion of the distal end member 1.

The distal end member 1 further includes a proximal end portion 18 positioned at an end of the bending portion 112 side of the distal end member 1 as illustrated in FIG. 3. An insulation ring 118 made of an insulation material is provided to the outer peripheral portion of the proximal end portion 18 as illustrated in FIG. 3 to FIG. 5. A resin material or a ceramic material, for example, is used for the insulation material forming the insulation ring 118.

The configuration of the first distal end cover 2 will be described next. The first distal end cover 2 includes a cylindrical portion 21 having a cylindrical shape with both ends opened, a first nozzle configuring portion 22 configuring at least part of the nozzle 4, and a coupling portion 23 coupling the cylindrical portion 21 and the first nozzle configuring portion 22 as illustrated in FIG. 2 to FIG. 4. The boundary among the cylindrical portion 21, the first nozzle configuring portion 22, and the coupling portion 23 is indicated by a dotted line in FIG. 3. The first nozzle configuring portion 22 configures part of the nozzle 4 and has a plate-like shape in the present embodiment. In addition, the first nozzle configuring portion 22 has an end surface 22a positioned at an end in a direction parallel to the central axis of the cylindrical portion 21.

The cylindrical portion 21 has a groove portion 21a to engage with part of the second distal end cover 3. The groove portion 21a is formed in an inner peripheral portion of the cylindrical portion 21 in a direction around the axis of the cylindrical portion 21. The coupling portion 23 couples the inner peripheral portion of the cylindrical portion 21 and the first nozzle configuring portion 22.

The configuration of the second distal end cover 3 will be described next. The second distal end cover 3 has a cylindrical shape covering the distal end member 1 as illustrated in FIG. 2 to FIG. 4. In addition, the second distal end cover 3 has a proximal end portion 31 and a distal end portion 32.

The second distal end cover 3 further has an opening portion 33 provided at the distal end portion 32 as illustrated in FIG. 2 to FIG. 4. The opening portion 33 is a portion through which part of the nozzle 4, the observation window 11, and the illumination window 12 are exposed, and the treatment instrument raised by the raising base 115 is lead out. The first nozzle configuring portion 22 of the first distal end cover 2 and the second nozzle configuring portion 14 of the distal end member 1 are exposed from the opening portion 33 as part of the nozzle 4 in the present embodiment.

The second distal end cover 3 further includes a restricting protrusion 34 to fit in the restricting recess 16 of the distal end member 1 as illustrated in FIG. 3 and FIG. 4. The restricting protrusion 34 is a protrusion formed in the inner peripheral portion of the second distal end cover 3 along the central axis of the second distal end cover 3.

The second distal end cover 3 further includes a fragile portion 35 as illustrated in FIG. 3 and FIG. 4. The fragile portion 35 is a portion that can be broken upon detaching the second distal end cover 3 from the distal end member 1. Hereinafter, a portion configuring the fragile portion 35 will specifically be described. A notch portion 36 formed along the central axis of the second distal end cover 3 is provided to the proximal end portion 31. In addition, a notch portion 37 formed along the central axis of the second distal end cover 3 in the vicinity of the notch portion 36 is provided to the distal end portion 32. The fragile portion 35 is configured by a portion between the notch portion 36 and the notch portion 37.

A plurality of notch portions other than the notch portion 36 is also provided to the proximal end portion 31 as illustrated in FIG. 3.

The second distal end cover 3 further includes an engagement hole 38, an engagement portion 39, and a joint opening 40 provided to the proximal end portion 31 as illustrated in FIG. 3 and FIG. 4. The engagement hole 38 is a hole to engage with the locking pin 17 of the distal end member 1. The engagement portion 39 is a portion to engage with the groove portion 21a provided to the first distal end cover 2. The joint opening 40 is an opening for passing through the first nozzle configuring portion 22 of the first distal end cover 2 upon attaching the first distal end cover 2 and the second distal end cover 3 to the distal end member 1. Note that the joint opening 40 is omitted in FIG. 3.

### (Procedure for Attaching First and Second Distal End Covers)

A procedure for attaching the first distal end cover 2 and the second distal end cover 3 to the distal end member 1 (hereinafter referred to as procedure for attaching) will be described next. In the procedure for attaching, firstly, the first nozzle configuring portion 22 of the first distal end cover 2 is passed through the joint opening 40 of the second distal end cover 3, and the engagement portion 39 of the second distal end cover 3 is engaged with the groove portion 21a of the first distal end cover 2. Thus, the first distal end cover 2 is joined to the second distal end cover 3 (see FIG. 3 and FIG. 4). FIG. 6 illustrates a state in which the first distal end cover 2 is joined to the second distal end cover 3.

The first nozzle configuring portion 22 of the first distal end cover 2 is covered by the distal end portion 32 of the second distal end cover 3 in the state illustrated in FIG. 6. Further, the proximal end portion 31 of the second distal end cover 3 (see FIG. 3 and FIG. 4) is covered by the cylindrical portion 21 of the first distal end cover 2.

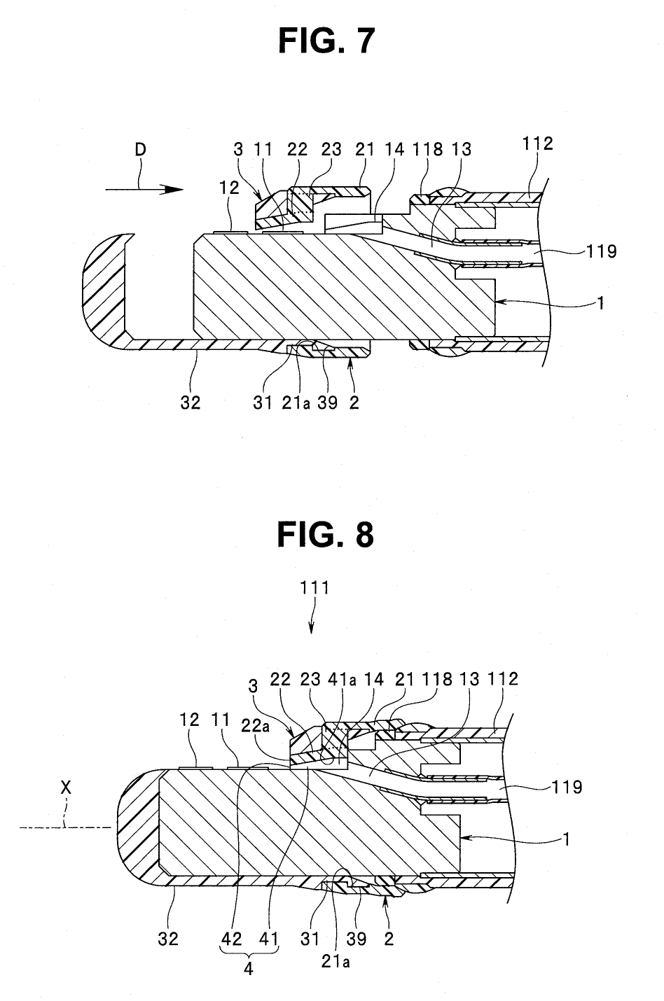

In the procedure for attaching, then, the restricting recess 16 of the distal end member 1 and the restricting protrusion 34 of the second distal end cover 3 are aligned with each other. While the restricting protrusion 34 is inserted into the restricting recess 16, the first and second distal end covers 2 and 3 integrated with each other are joined to the distal end member 1 (see FIG. 3 to FIG. 5). FIG. 7 illustrates a midway state of attaching the first and second distal end covers 2 and 3 to the distal end member 1. An arrow denoted by symbol D indicates an insertion direction of the first and second distal end covers 2 and 3 in FIG. 7.

The restricting recess 16 and the restricting protrusion 34 have a function to restrict the postures of the first and second distal end covers 2 and 3. The function prevents the rotation of the first and second distal end covers 2 and 3 in the direction around the insertion axis X of the insertion portion 110 illustrated in FIG. 2 upon attaching the first and second distal end covers 2 and 3 to the distal end member 1.

In the procedure for attaching, then, the first and second distal end covers 2 and 3 are pushed toward the arrow D direction from the state illustrated in FIG. 7 and the engagement hole 38 of the second distal end cover 3 is engaged with the locking pin 17 of the distal end member 1 (see FIG. 3 and FIG. 4). Thus, the first and second distal end covers 2 and 3 are fixed to the distal end member 1, and the attachment of the first and second distal end covers 2 and 3 is completed. Further, the first nozzle configuring portion 22 of the first distal end cover 2 is in close contact with the second nozzle configuring portion 14 of the distal end member 1 when the first and second distal end covers 2 and 3 are attached to the distal end member 1 (see FIG. 3 to FIG. 5). Thus, attachment of the nozzle 4 is completed.

Hereinafter, a state in which the first distal end cover 2 and the second distal end cover 3 are attached to the distal end member 1 will be referred to as a “mounted state”. FIG. 2 and FIG. 8 illustrate a normal mounted state. The nozzle 4 is normally formed in the state.

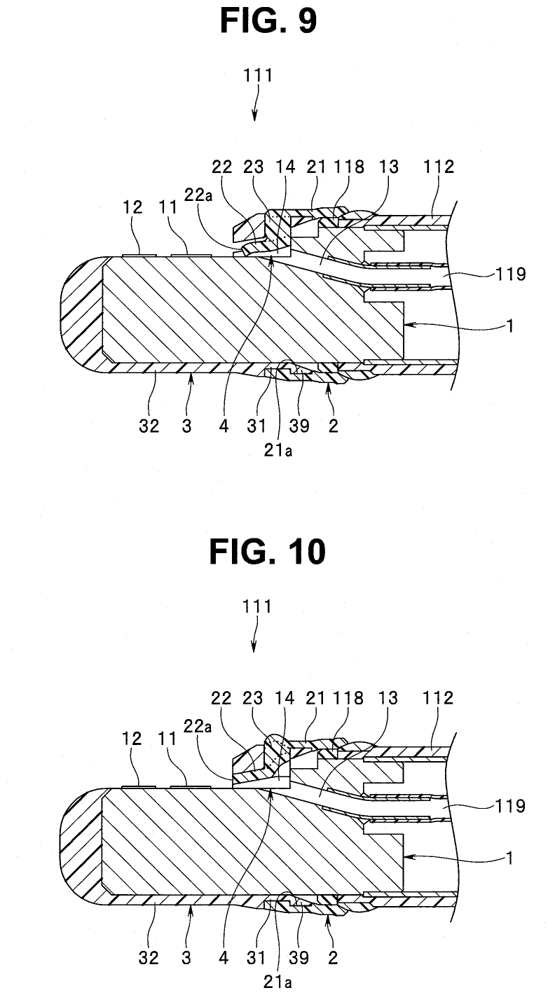

FIG. 9 illustrates a first example of an abnormal mounted state. FIG. 10 illustrates a second example of the abnormal mounted state. In both of the first and second examples, the first nozzle configuring portion 22 is deformed, and the nozzle 4 is not normally formed. In the first example, the end surface 22a of the first nozzle configuring portion 22 sinks toward the back side (right side in FIG. 9) of the nozzle 4. In the second example, the first nozzle configuring portion 22 deforms in the back side of the nozzle 4, and as a result, there rises the outer peripheral portion of the cylindrical portion 21 of the first distal end cover 2.

Note that the first nozzle configuring portion 22 of the first distal end cover 2 is covered by the second distal end cover 3 in the mounted state. The cylindrical portion 21 of the first distal end cover 2 covers a portion where the locking pin 17 of the distal end member 1 and the engagement hole 38 of the second distal end cover 3 are engaged with each other (see FIG. 3 and FIG. 4), and protects the portion in the mounted state. The cylindrical portion 21 is in close contact with the insulation ring 118 in the mounted state (see FIG. 3 to FIG. 5). In addition, part of the first distal end cover 2 is exposed in the mounted state. The cylindrical portion 21 and the end surface 22a which is part of the first nozzle configuring portion 22 are exposed as part of the first distal end cover 2 in the present embodiment.

### (Configuration of Nozzle)

The configuration of the nozzle 4 will be described in detail with reference to FIG. 2 and FIG. 8 next. The nozzle 4 includes a nozzle channel portion 41 communicating with the fluid passage 13 of the distal end member 1 and an ejection port 42 positioned at the end of the nozzle channel portion 41 in the mounted state. The ejection port 42 ejects a predetermined fluid to the outside. The predetermined fluid is blown to the observation window 11 and the illumination window 12.

At least part of the ejection port 42 is configured by the first nozzle configuring portion 22. Both of the nozzle channel portion 41 and the ejection port 42 are configured by the first nozzle configuring portion 22 and the second nozzle configuring portion 14 in the present embodiment.

The nozzle channel portion 41 includes a channel bending portion 41a as a bending portion having a bent shape as illustrated in FIG. 8. The traveling direction of a fluid flowing through the nozzle channel portion 41 changes in the channel bending portion 41a. Here, a first channel through which the fluid travels straight from the fluid passage 13 toward the channel bending portion 41a, and a second channel through which the fluid travels straight from the channel bending portion 41a toward the ejection port 42 are assumed in the nozzle channel portion 41. The angle formed by the central axis of the second channel with respect to a virtual straight line including the central axis of the first channel is an acute angle in the example illustrated in FIG. 8. This angle is 30°, for example.

The first nozzle configuring portion 22 configures at least part of the channel bending portion 41a. Part of the first nozzle configuring portion 22 has a plane that is part of the inner peripheral surface of the nozzle channel portion 41 and configures an upper plane in FIG. 8 in the present embodiment. Hereinafter, the upper plane of the nozzle channel portion 41 in FIG. 8 will be referred to as a top plane. Further, the plane of the first nozzle configuring portion 22 configuring the top plane of the nozzle channel portion 41 will be referred to as the top plane. The top plane of the first nozzle configuring portion 22 includes a bending portion having a bent shape. The channel bending portion 41a is configured by a bending portion of the top plane of the first nozzle configuring portion 22.

The first nozzle configuring portion 22 is sandwiched between the second distal end cover 3 and the second nozzle configuring portion 14 and the position thereof is restricted in the mounted state as illustrated in FIG. 2 and FIG. 8. At least part of the first nozzle configuring portion 22 is positioned between the second distal end cover 3 and the second nozzle configuring portion 14 in a cross-section perpendicular to the insertion axis X of the insertion portion 110 in the mounted state, particularly in the present embodiment.

### (Procedure for Detaching First and Second Distal End Covers)

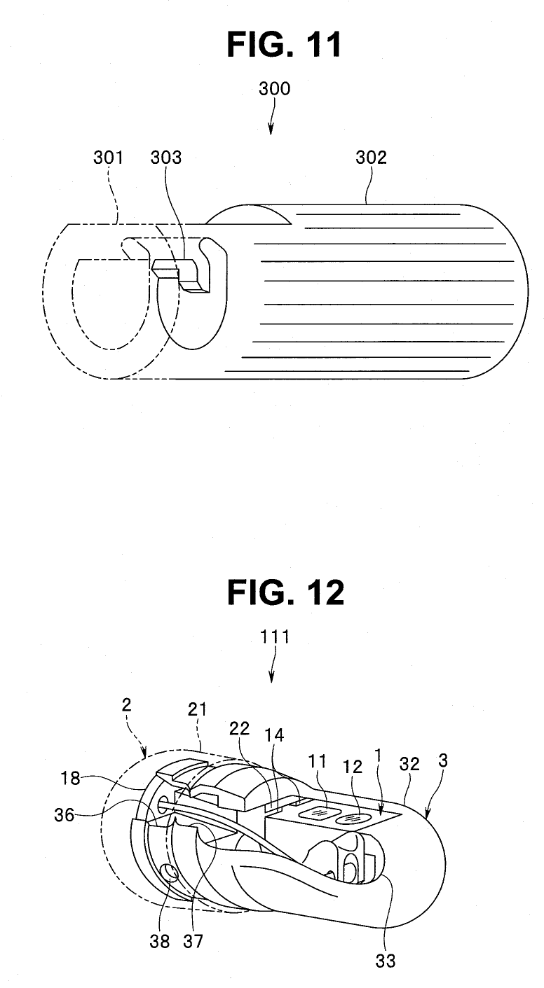

A procedure for detaching the first distal end cover 2 and the second distal end cover 3 from the distal end member 1 (hereinafter referred to as “procedure for detaching”) will be described next. A detaching jig 300 illustrated in FIG. 11 is used in the procedure for detaching. The detaching jig 300 includes a cylindrical portion 301 having a cylindrical shape with one end opened, a grasping portion 302 coupled to the cylindrical portion 301, and a claw portion 303 that is a protrusion formed inside the cylindrical portion 301. The cylindrical portion 301 is depicted by a dashed-and-double-dotted line in FIG. 11. The inner diameter of the cylindrical portion 301 is larger than the outer diameter of the distal end side portion of the distal end portion 32 of the second distal end cover 3. The claw portion 303 is configured to be hooked to an opening portion 33 provided to the distal end portion 32 of the second distal end cover 3.

Hereinafter, the procedure for detaching will be described with reference to FIG. 2, FIG. 3, and FIG. 11. In the procedure for detaching, firstly, the distal end portion 111 is covered by the cylindrical portion 301 of the detaching jig 300 such that the claw portion 303 of the detaching jig 300 is hooked to the opening portion 33 of the second distal end cover 3. The detaching jig 300 is rotated in the direction around the insertion axis X of the insertion portion 110 in a state where the claw portion 303 is hooked to the opening portion 33 next. The rotation direction of the detaching jig 300, when the distal end portion 111 is viewed in a direction parallel to the insertion axis X from the distal end side of the insertion portion 110, is a counterclockwise direction, for example. With this, the portion between the notch portion 36 and the notch portion 37 in the second distal end cover 3, that is, the fragile portion 35 is broken. FIG. 12 illustrates a state in which the fragile portion 35 is broken.

When the fragile portion 35 is broken, the engagement hole 38 of the second distal end cover 3 is disengaged from the locking pin 17 of the distal end member 1. In the procedure for detaching, then, the detaching jig 300 is removed from the distal end portion 111. The first distal end cover 2 and the second distal end cover 3 in which the fragile portion 35 is broken are pulled out from the distal end member 1. Thus, the first distal end cover 2 and the second distal end cover 3 are detached from the distal end member 1.

### (Action and Effect)

The action and the effect of the endoscope 100 according to the present embodiment will be described next. The endoscope 100 according to the present embodiment includes the first distal end cover 2 made of an elastically deformable material, and the first nozzle configuring portion 22 provided to the first distal end cover 2. The first nozzle configuring portion 22 configures the channel bending portion 41a having a bent shape in the nozzle channel portion 41 of the nozzle 4 in the present embodiment. The first nozzle configuring portion 22 is covered by the second distal end cover 3 made of a material harder than that of the first distal end cover 2 in the mounted state. With this, according to the present embodiment, the first distal end cover 2 may be prevented from being deformed as compared with a case where the first distal end cover 2 is not covered by another member or a case where the second distal end cover 3 is made of the same material as that of the first distal end cover 2. That is, according to the present embodiment, a deformation of the first nozzle configuring portion 22 caused by pressure or impact generated by a fluid may be prevented. The pressure or impact is generated when a fluid such as cleaning water or air passes through the channel bending portion 41a. Thus, according to the present embodiment, a decrease of the amount of fluid ejected from the nozzle 4 may be prevented, and deterioration of a cleaning ability for the observation window 11 and the illumination window 12 may be prevented.

The first nozzle configuring portion 22 also configures part of the ejection port 42 of the nozzle 4, particularly in the present embodiment. As described above, according to the present embodiment, the deformation of the ejection port 42 may be prevented by preventing the deformation of the first nozzle configuring portion 22. As a result, according to the present embodiment, the deviation of the direction of the fluid ejected from the ejection port 42 may be prevented, whereby deterioration of the cleaning ability for the observation window 11 and the illumination window 12 may be prevented.

The endoscope 100 according to the present embodiment further includes the second nozzle configuring portion 14 that is provided to the distal end member 1 and configures another part of the nozzle 4. The nozzle channel portion 41 and the ejection port 42 of the nozzle 4 are configured by the first nozzle configuring portion 22 and the second nozzle configuring portion 14 in the present embodiment. The first nozzle configuring portion 22 is sandwiched between the second distal end cover 3 and the second nozzle configuring portion 14, and the position thereof is restricted in the mounted state. The first distal end cover 2 is formed of an elastically deformable material in the present embodiment. This configuration allows the first nozzle configuring portion 22 to be in close contact with the second distal end cover 3 and the second nozzle configuring portion 14. With this, according to the present embodiment, it is possible to prevent the fluid from leaking from the gap between the first nozzle configuring portion 22 and the second distal end cover 3, and between the first nozzle configuring portion 22 and the second nozzle configuring portion 14.

In addition, the fragile portion 35 of the second distal end cover 3 is to be broken to detach the first and second distal end covers 2 and 3 from the distal end member 1 in the present embodiment. With this, according to the present embodiment, an unnecessary external force may be prevented from applying to the second nozzle configuring portion 14 upon detaching the second distal end cover 3 from the distal end member 1, and as a result, deformation or breakage of the second nozzle configuring portion 14 may be prevented.

Note that an unnecessary external force is less likely to apply to the second nozzle configuring portion 14 upon detaching the first distal end cover 2 from the distal end member 1, since the first distal end cover 2 is formed of an elastically deformable material in the present embodiment.

Part of the first distal end cover 2, specifically the cylindrical portion 21 and the end surface 22a of the first nozzle configuring portion 22 of the first distal end cover 2 are exposed in the mounted state in the present embodiment. In the abnormal mounted state, the position or shape of part of the first distal end cover 2 changes from that in the normal mounted state illustrated in FIG. 8, as illustrated in FIG. 9 and FIG. 10. According to the present embodiment, therefore, it is possible to check whether or not the first and second distal end covers 2 and 3 are normally attached to the distal end member 1 by observing part of the exposed first distal end cover 2 in the mounted state. With this, according to the present embodiment, it is possible to check whether or not the nozzle 4 is normally formed.

Meanwhile, in a case where a nozzle for cleaning the observation window 11 and the illumination window 12 is provided separately from the first and second distal end covers 2 and 3, problems may arise. The problems include to forget to detach the nozzle from the endoscope 100 after use, or to forget to clean the fluid passage 13 because of having forgotten to detach the nozzle. In contrast, in the present embodiment, the nozzle 4 is configured by the first nozzle configuring portion 22 provided to the first distal end cover 2 and the second nozzle configuring portion 14 provided to the distal end member 1. With this, according to the present embodiment, the occurrence of the above-described problems may be prevented. Further, according to the present embodiment, the number of components may be reduced as compared with the above-described case and the number of work steps may be reduced by omitting the work for attaching the nozzle.

### Second Embodiment

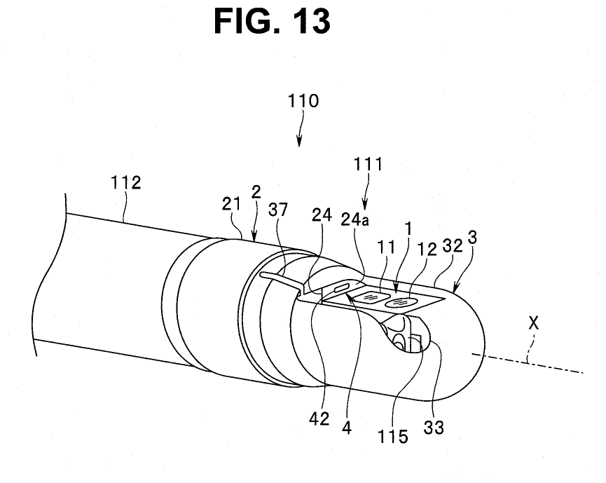

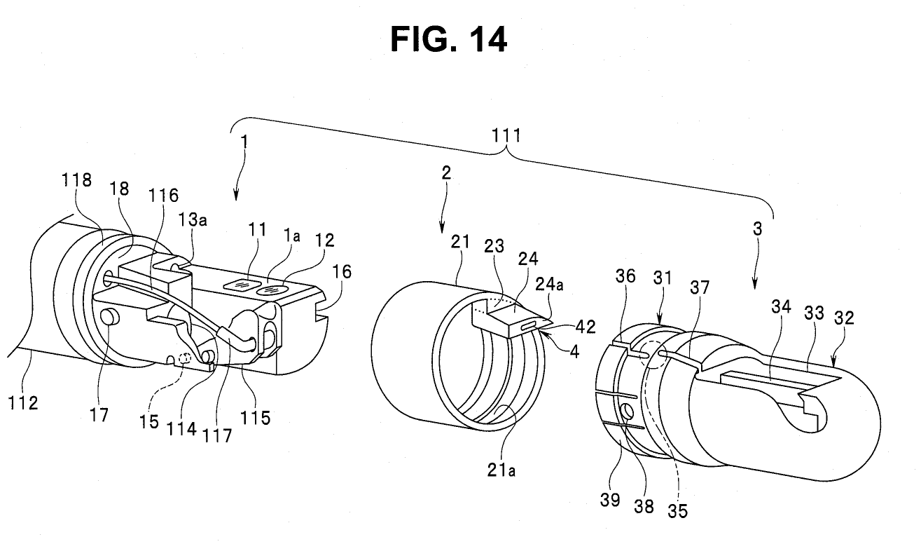

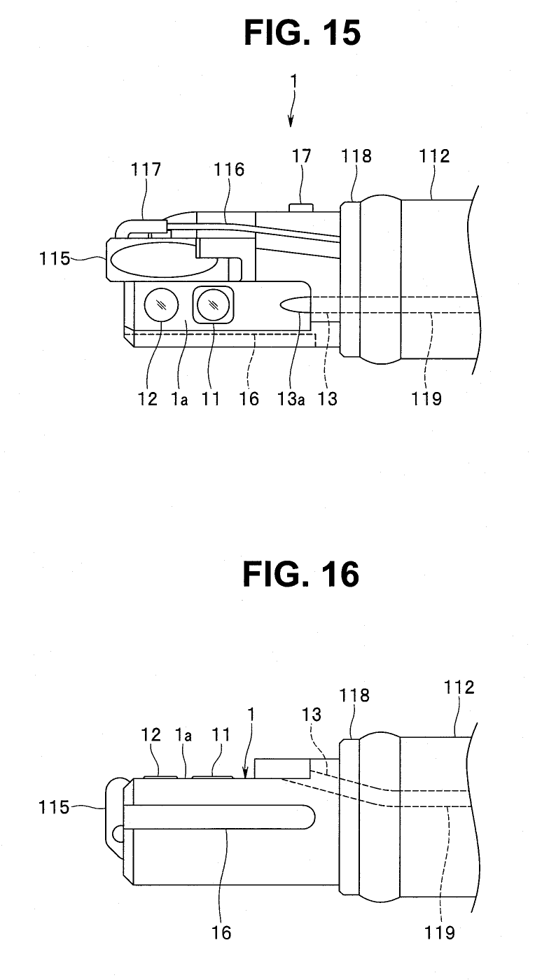

A second embodiment of the present invention will be described next. First, a configuration of an endoscope 100 according to the present embodiment will be described with reference to FIG. 13 to FIG. 16. FIG. 13 is a perspective view of a distal end portion according to the present embodiment. FIG. 14 is a perspective view of the disassembled distal end portion illustrated in FIG. 13. FIG. 15 is a plan view of a distal end member according to the present embodiment. FIG. 16 is a side view of the distal end member according to the present embodiment.

The configuration of the endoscope 100 according to the present embodiment is different from that of the first embodiment in following points. The second nozzle configuring portion 14 in the first embodiment is not provided to the distal end member 1 in the present embodiment. Further, the first distal end cover 2 includes a first nozzle configuring portion 24 instead of the first nozzle configuring portion 22 in the first embodiment. The nozzle 4 according to the present embodiment is entirely configured by the first nozzle configuring portion 24.

In addition, the coupling portion 23 of the first distal end cover 2 couples the inner peripheral portion of the cylindrical portion 21 of the first distal end cover 2 and the first nozzle configuring portion 24 in the present embodiment. The boundary between the cylindrical portion 21, the coupling portion 23, and the first nozzle configuring portion 24 is indicated by a dotted line in FIG. 14. The first nozzle configuring portion 24 has an end surface 24a that is farthest from the cylindrical portion 21.

The insertion axis X of the insertion portion 110 (central axis of insertion portion 110) is indicated by a dashed-and-dotted line in FIG. 13. In FIG. 14, depicted are the distal end member 1, the first distal end cover 2, and the second distal end cover 3 separated from each other in a direction parallel to the insertion axis X illustrated in FIG. 13. The configuration of the nozzle 4 according to the present embodiment will be described later.

The procedure for attaching according to the present embodiment will be briefly described next. The procedure for attaching according to the present embodiment is basically the same as the procedure for attaching in the first embodiment. However, in the present embodiment, when the first distal end cover 2 is joined to the second distal end cover 3, the first nozzle configuring portion 24 of the first distal end cover 2 is passed through the joint opening 40 of the second distal end cover 3 (see FIG. 4), and then, the first distal end cover 2 is joined to the second distal end cover 3.

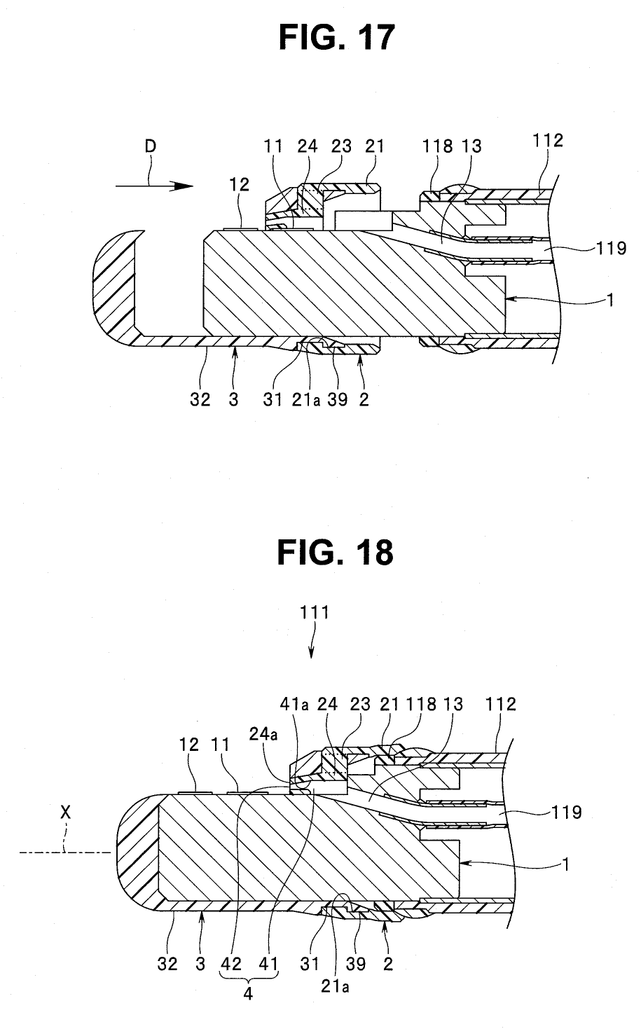

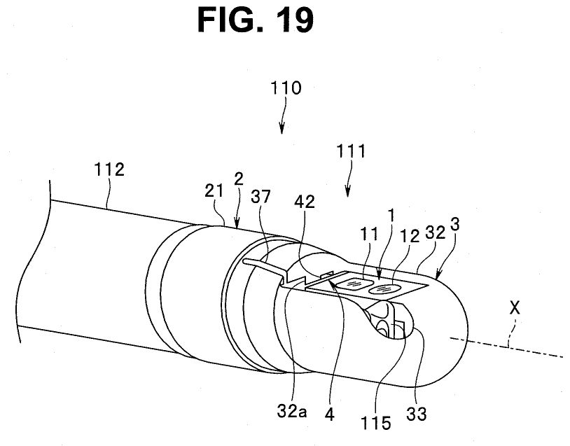

FIG. 17 illustrates a midway state of attaching the first and second distal end covers 2 and 3 to the distal end member 1. FIG. 13 and FIG. 18 illustrate a state in which the first distal end cover 2 and the second distal end cover 3 are attached to the distal end member 1, that is, the mounted state.

The configuration of the nozzle 4 according to the present embodiment will be described with reference to FIG. 18 next. As described in the first embodiment, the nozzle 4 includes the nozzle channel portion 41 communicating with the fluid passage 13 of the distal end member 1 and the ejection port 42 positioned at the end of the nozzle channel portion 41 in the mounted state. The entire nozzle channel portion 41 is configured by a conduit formed in the first nozzle configuring portion 24 of the first distal end cover 2 in the present embodiment. The conduit in the first nozzle configuring portion 24 communicates with the fluid passage 13 in the mounted state. In addition, the ejection port 42 is formed on the end surface 24a of the first nozzle configuring portion 24 in the present embodiment.

As described in the first embodiment, the nozzle channel portion 41 includes the channel bending portion 41a having a bent shape. The entire channel bending portion 41a is configured by the above-described conduit in the first nozzle configuring portion 24 in the present embodiment.

The first nozzle configuring portion 24 is covered by the second distal end cover 3 in the mounted state as illustrated in FIG. 13 and FIG. 18. The first nozzle configuring portion 24 is sandwiched between the second distal end cover 3 and the distal end member 1, and the position thereof is restricted in the mounted state. At least part of the first nozzle configuring portion 24 is positioned between the second distal end cover 3 and the distal end member 1 in a cross-section perpendicular to the insertion axis X of the insertion portion 110 in the mounted state, particularly in the present embodiment.

Other configurations, actions, and effects in the present embodiment are the same as those in the first embodiment.

### Third Embodiment

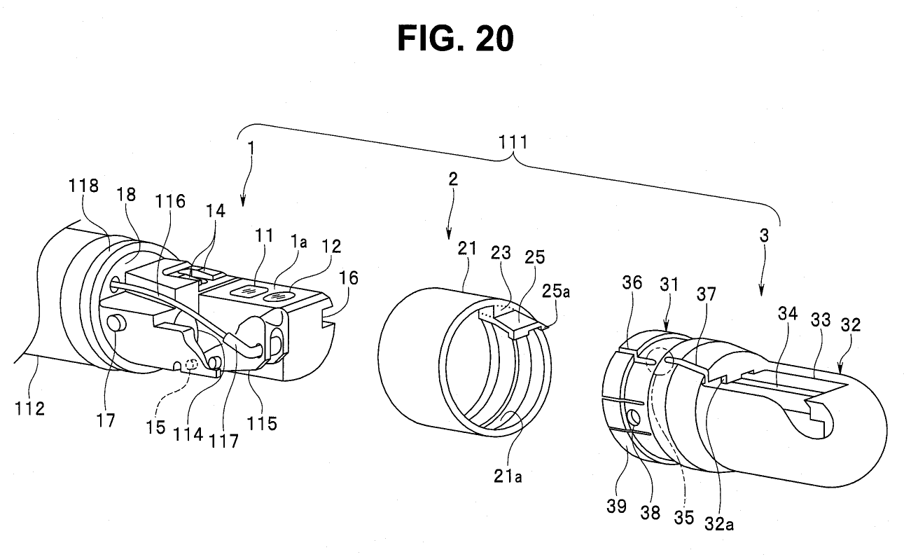

A third embodiment of the present invention will be described next. First, a configuration of the endoscope 100 according to the present embodiment will be described with reference to FIG. 19 and FIG. 20. FIG. 19 is a perspective view of a distal end portion according to the present embodiment. FIG. 20 is a perspective view of the disassembled distal end portion illustrated in FIG. 19.

The configuration of the endoscope 100 according to the present embodiment is different from that of the first embodiment in following points. In the present embodiment, the first distal end cover 2 includes a first nozzle configuring portion 25 instead of the first nozzle configuring portion 22 in the first embodiment. The first nozzle configuring portion 25 configures part of the nozzle 4 in the present embodiment.

In addition, the coupling portion 23 of the first distal end cover 2 couples the inner peripheral portion of the cylindrical portion 21 of the first distal end cover 2 and the first nozzle configuring portion 25 in the present embodiment. The boundary between the cylindrical portion 21, the coupling portion 23, and the first nozzle configuring portion 25 is indicated by a dotted line in FIG. 20. The first nozzle configuring portion 25 has an end surface 25a positioned at an end in a direction parallel to the central axis of the cylindrical portion 21.

The endoscope 100 according to the present embodiment includes a third nozzle configuring portion 32a provided to the second distal end cover 3 and configuring another part of the nozzle 4 in the present embodiment. The third nozzle configuring portion 32a is part of the distal end portion 32 of the second distal end cover 3.

The insertion axis X of the insertion portion 110 (central axis of insertion portion 110) is indicated by a dashed-and-dotted line in FIG. 19. In FIG. 20, depicted are the distal end member 1, the first distal end cover 2, and the second distal end cover 3 separated from each other in a direction parallel to the insertion axis X illustrated in FIG. 19. The configuration of the nozzle 4 according to the present embodiment will be described later.

The procedure for attaching according to the present embodiment will be briefly described next. The procedure for attaching according to the present embodiment is basically the same as the procedure for attaching in the first embodiment. However, in the present embodiment, when the first distal end cover 2 is joined to the second distal end cover 3, the first nozzle configuring portion 25 of the first distal end cover 2 is passed through the joint opening 40 of the second distal end cover 3 (see FIG. 4), and then, the first distal end cover 2 is joined to the second distal end cover 3.

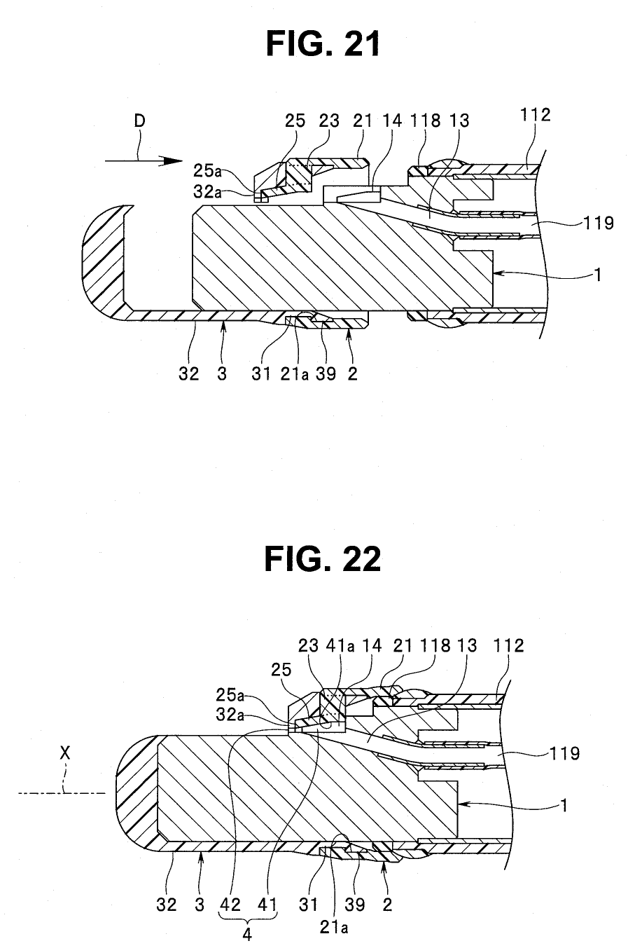

FIG. 21 illustrates a midway state of attaching the first and second distal end covers 2 and 3 to the distal end member 1. FIG. 19 and FIG. 22 illustrate a state in which the first distal end cover 2 and the second distal end cover 3 are attached to the distal end member 1, that is, the mounted state.

Part of the first distal end cover 2 is exposed in the mounted state. The cylindrical portion 21 is exposed as part of the first distal end cover 2 in the present embodiment. Further, the third nozzle configuring portion 32a of the second distal end cover 3 is configured by a portion of the distal end portion 32 that is in contact with the end surface 25a of the first nozzle configuring portion 25 in the mounted state.

The configuration of the nozzle 4 according to the present embodiment will be described with reference to FIG. 20 and FIG. 22 next. As described in the first embodiment, the nozzle 4 includes the nozzle channel portion 41 communicating with the fluid passage 13 of the distal end member 1 and the ejection port 42 positioned at the end of the nozzle channel portion 41 in the mounted state. The nozzle channel portion 41 is configured by the first nozzle configuring portion 25 of the first distal end cover 2, the second nozzle configuring portion 14 of the distal end member 1, and the third nozzle configuring portion 32a of the second distal end cover 3 in the present embodiment.

In addition, the ejection port 42 is configured by the third nozzle configuring portion 32a in the present embodiment.

As described in the first embodiment, the nozzle channel portion 41 includes the channel bending portion 41a having a bent shape. The first nozzle configuring portion 25 has a plane part of which configures the top plane of the nozzle channel portion 41 in the present embodiment. Hereinafter, also the plane of the first nozzle configuring portion 25 configuring the top plane of the nozzle channel portion 41 will be referred to as the top plane. The top plane of the first nozzle configuring portion 25 includes a bending portion having a bent shape. The channel bending portion 41a is configured by the bending portion of the top plane of the first nozzle configuring portion 25.

The first nozzle configuring portion 25 is covered by the second distal end cover 3 in the mounted state as illustrated in FIG. 22. In addition, the first nozzle configuring portion 25 is sandwiched between the second distal end cover 3 and the second nozzle configuring portion 14, and the position thereof is restricted in the mounted state. At least part of the first nozzle configuring portion 25 is positioned between the second distal end cover 3 and the second nozzle configuring portion 14 in a cross-section perpendicular to the insertion axis X of the insertion portion 110 in the mounted state, particularly in the present embodiment.

Other configurations, actions, and effects in the present embodiment are the same as those in the first embodiment.

### Other Embodiments

Incidentally, the description has been made on a side viewing endoscope or an oblique viewing endoscope for an example in the above-described embodiments. The same structure as that of the fragile portion 35 in each of the above embodiments can also be applied to a distal end cover used for a forward viewing endoscope. Hereinafter, a description will be given of an embodiment relating to a forward viewing endoscope.

### (Configuration of Endoscope)

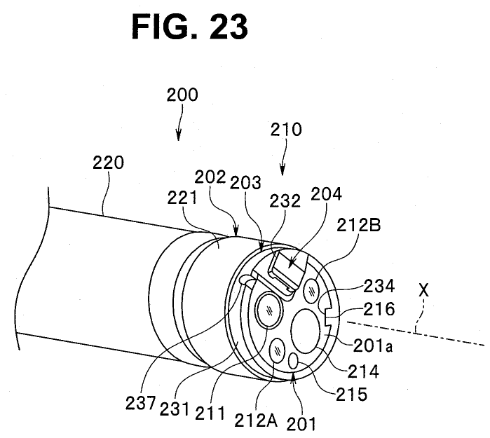

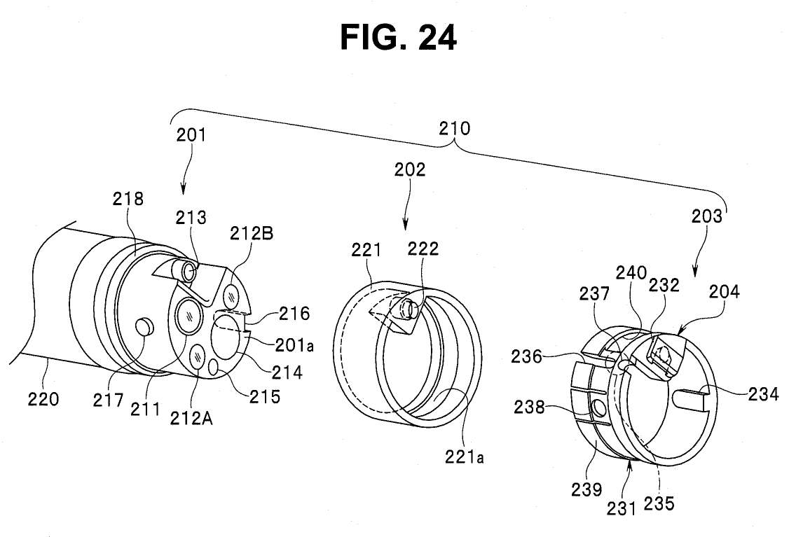

First, the configuration of the endoscope will be described with reference to FIG. 23 and FIG. 24. FIG. 23 is a perspective view illustrating a distal end portion of a forward viewing endoscope. FIG. 24 is a perspective view of the disassembled distal end portion illustrated in FIG. 23. The endoscope illustrated in FIG. 23 includes an insertion portion 200 to be inserted into a subject. The insertion portion 200 has an elongated shape, and includes a distal end portion 210 that is positioned at a distal end of the insertion portion 200, a bending portion 220 that is bendable, and a flexible tube portion that has flexibility. The distal end portion 210, the bending portion 220, and the flexible tube portion are connected to each other in this order from the distal end side of the insertion portion 200.

The distal end portion 210 is provided with an image pickup optical system and an illumination optical system that are optical members for observation, an image pickup apparatus, a treatment instrument outlet port 214 that is an opening portion of a treatment instrument channel provided in the insertion portion 200, and a forward water feeding port 215. The image pickup apparatus includes an image pickup device that picks up an image of a subject.

The endoscope further includes a distal end member 201, a first distal end cover 202, a second distal end cover 203, and a nozzle 204 that configure the distal end portion 210. The distal end member 201 constitutes the main portion of the distal end portion 210. The first distal end cover 202 and the second distal end cover 203 are attached to the distal end member 201. The distal end member 201 is made of a metal material such as stainless steel, for example.

The insertion axis X of the insertion portion 200 (central axis of insertion portion 200) is indicated by a dashed-and-dotted line in FIG. 23. In FIG. 24, depicted are the distal end member 201, the first distal end cover 202, and the second distal end cover 203 separated from each other in a direction parallel to the insertion axis X illustrated in FIG. 23.

The first distal end cover 202 is made of an elastically deformable material, and is detachably attached to the distal end member 201. The second distal end cover 203 is made of a material harder than that of the first distal end cover 202, and is attached to the distal end member 201. The first distal end cover 202 is formed of the same material as that of the first distal end cover 2 in the first embodiment, for example. The second distal end cover 203 is formed of the same material as that of the second distal end cover 3 in the first embodiment, for example.

The distal end member 201 has a substantially columnar shape extending in the direction of the insertion axis X of the insertion portion 200, and has an end surface 201a perpendicular to the insertion axis X of the insertion portion 200 as illustrated in FIG. 23 and FIG. 24. On the end surface 201a of the distal end member 201, provided are an observation window 211 positioned at a distalmost end of the image pickup optical system, and two illumination windows 212A and 212B positioned at a distalmost end of the illumination optical system.

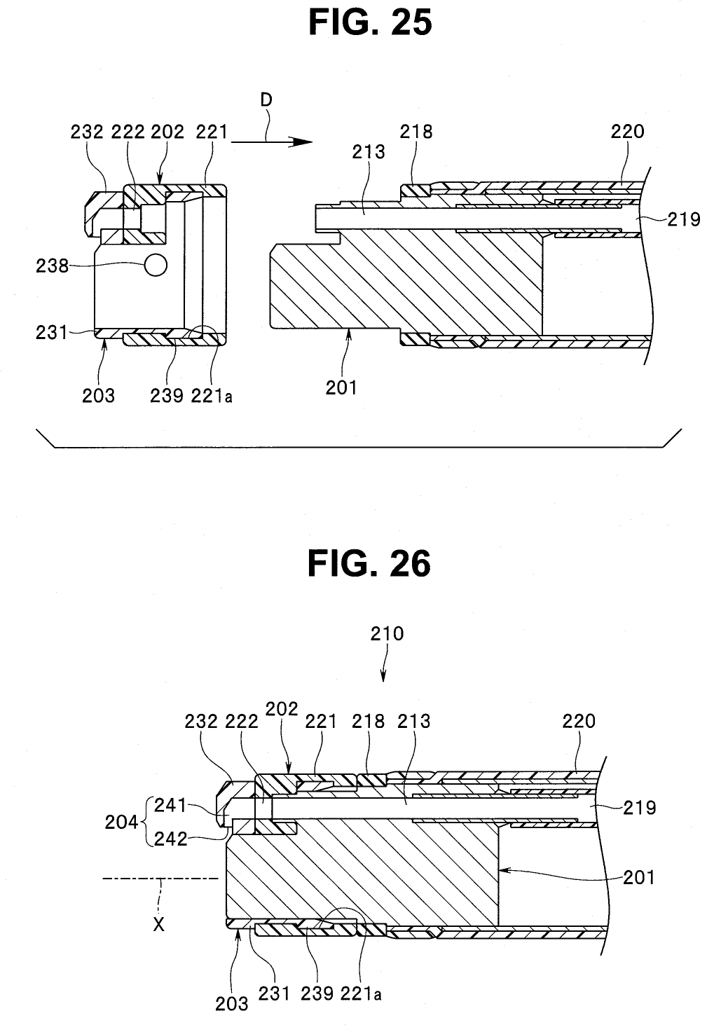

The nozzle 204 serves to clean the surface of the observation window 211, and is configured to be able to blow a predetermined fluid to the observation window 211. The predetermined fluid is cleaning water and air, for example. The distal end member 201 further includes a fluid passage 213 for allowing the predetermined fluid to flow through as illustrated in FIG. 24. The fluid passage 213 communicates with an air/water feeding conduit 219 provided inside the insertion portion 200. Note that the air/water feeding conduit 219 is illustrated in such as FIG. 25 which will be described later.

The distal end member 201 further includes a restricting recess 216 and a locking pin 217 as illustrated in FIG. 24. The restricting recess 216 and the locking pin 217 are provided in the outer peripheral portion of the distal end member 201. The restricting recess 216 is a groove portion formed in the outer peripheral portion of the distal end member 201 along the insertion axis X of the insertion portion 200. The locking pin 217 is a protrusion protruding from the outer peripheral portion of the distal end member 201.

An insulation ring 218 made of an insulation material is provided at the proximal end portion of the distal end member 201 as illustrated in FIG. 24. The insulation ring 218 is formed of the same material as that of the insulation ring 118 in the first embodiment, for example.

The first distal end cover 202 includes a cylindrical portion 221 having a cylindrical shape with both ends opened, and a channel connection portion 222 communicating with the fluid passage 213 in a mounted state described later, as illustrated in FIG. 24. The cylindrical portion 221 includes a groove portion 221a to engage with part of the second distal end cover 203. The groove portion 221a is formed in an inner peripheral portion of the cylindrical portion 221 in the direction around the axis of the cylindrical portion 221.

The second distal end cover 203 includes a cylindrical portion 231 having a cylindrical shape with both ends opened, and a nozzle configuring portion 232 coupled to the cylindrical portion 231 as illustrated in FIG. 24. In the present embodiment, the entire nozzle 204 is configured by the nozzle configuring portion 232. The configuration of the nozzle 204 will be described later.

The second distal end cover 203 further includes a restricting protrusion 234 to fit in the restricting recess 216 of the distal end member 201 as illustrated in FIG. 23 and FIG. 24. The restricting protrusion 234 is a protrusion formed in the inner peripheral portion of the second distal end cover 203 along the central axis of the second distal end cover 203.

The second distal end cover 203 further includes a fragile portion 235 as illustrated in FIG. 24. The fragile portion 235 is a portion that can be broken upon detaching the second distal end cover 203 from the distal end member 201. Hereinafter, a portion constituting the fragile portion 235 will specifically be described. The second distal end cover 203 is provided with two notch portions 236 and 237 formed along the central axis of the second distal end cover 203 and arranged side by side along the central axis. The fragile portion 235 is configured by a portion between the notch portion 236 and the notch portion 237.

The second distal end cover 203 further includes an engagement hole 238, an engagement portion 239, and a joint opening 240 as illustrated in FIG. 24. The engagement hole 238 is a hole to engage with the locking pin 217 of the distal end member 201. The engagement portion 239 is a portion having a relatively large outer diameter in the second distal end cover 203, and is a portion to engage with the groove portion 221a provided to the first distal end cover 202. The joint opening 240 is an opening for passing through the channel connection portion 222 of the first distal end cover 202 and the portion in the vicinity thereof upon attaching the first distal end cover 202 and the second distal end cover 203 to the distal end member 201.

### (Procedure for Attaching First and Second Distal End Covers)

A procedure for attaching the first distal end cover 202 and the second distal end cover 203 to the distal end member 201 (hereinafter referred to as procedure for attaching) will be described next. In the procedure for attaching, firstly, the channel connection portion 222 of the first distal end cover 202 and the vicinity portion thereof is passed through the joint opening 240 of the second distal end cover 203, and the engagement portion 239 of the second distal end cover 203 is engaged with the groove portion 221a provided to the first distal end cover 202. Thus, the first distal end cover 202 is joined to the second distal end cover 203 (see FIG. 24).

Next, the restricting recess 216 of the distal end member 201 and the restricting protrusion 234 of the second distal end cover 203 are aligned, and the first and second distal end covers 202 and 203 integrated with each other are joined to the distal end member 201 while inserting the restricting protrusion 234 into the restricting recess 216 (see FIG. 24). FIG. 25 illustrates a state before the first and second distal end covers 202 and 203 are attached to the distal end member 201. An arrow denoted by symbol D indicates an insertion direction of the first and second distal end covers 202 and 203 in FIG. 25.

The restricting recess 216 and the restricting protrusion 234 have a function to restrict the postures of the first and second distal end covers 202 and 203. The function prevents the rotation of the first and second distal end covers 202 and 203 in the direction around the insertion axis X of the insertion portion 200 illustrated in FIG. 23 upon attaching the first and second distal end covers 202 and 203 to the distal end member 201.

In the procedure for attaching, then, the joined first and second distal end covers 202 and 203 are pushed to the distal end member 201 in the arrow D direction illustrated in FIG. 25, and the engagement hole 238 of the second distal end cover 203 is engaged with the locking pin 217 of the distal end member 201 (see FIG. 24). Thus, the first and second distal end covers 202 and 203 are fixed to the distal end member 201, and the attachment of the first and second distal end covers 202 and 203 is completed. FIG. 23 and FIG. 26 illustrate a state in which the first distal end cover 202 and the second distal end cover 203 are attached to the distal end member 201, that is, the mounted state.

Note that the cylindrical portion 221 of the first distal end cover 202 covers the portion where the locking pin 217 of the distal end member 201 and the engagement hole 238 of the second distal end cover 203 are engaged with each other, and protects the portion in the mounted state. The cylindrical portion 221 is in close contact with the insulation ring 218 (see FIG. 24) in the mounted state.

### (Configuration of Nozzle)

The configuration of the nozzle 204 will be described in detail with reference to FIG. 26 next. The nozzle 204 has a nozzle channel portion 241 and an ejection port 242 positioned at the end of the nozzle channel portion 241. The nozzle channel portion 241 includes a portion having a bent shape and communicates with the channel connection portion 222 of the first distal end cover 202 in the mounted state. The channel connection portion 222 communicates with the fluid passage 213 of the distal end member 201 in the mounted state. The ejection port 242 ejects a predetermined fluid to the outside. The predetermined fluid is blown to the observation window 211.

The nozzle channel portion 241 is connected to the fluid passage 213 of the distal end member 201 via the first distal end cover 202 made of an elastically deformable material in the mounted state as illustrated in FIG. 26. The first distal end cover 202 is in close contact with the fluid passage 213 and the periphery of the nozzle channel portion 241, whereby the leakage of the fluid may be prevented.

Note that the entire nozzle 204 is configured by the nozzle configuring portion 232 which is part of the second distal end cover 203 in the present embodiment. In a case where the second distal end cover 203 is formed of an elastically deformable material similarly to the first distal end cover 202, the nozzle configuring portion 232, that is, the nozzle 204 may deform because of the pressure and impact of a fluid when the fluid such as cleaning water or air passes through the nozzle channel portion 241. In contrast, the second distal end cover 203 is made of a material harder than that of the first distal end cover 202 in the present embodiment. With this, according to the present embodiment, the deforming of the nozzle 204 may be prevented.

### (Procedure for Detaching First and Second Distal End Covers)

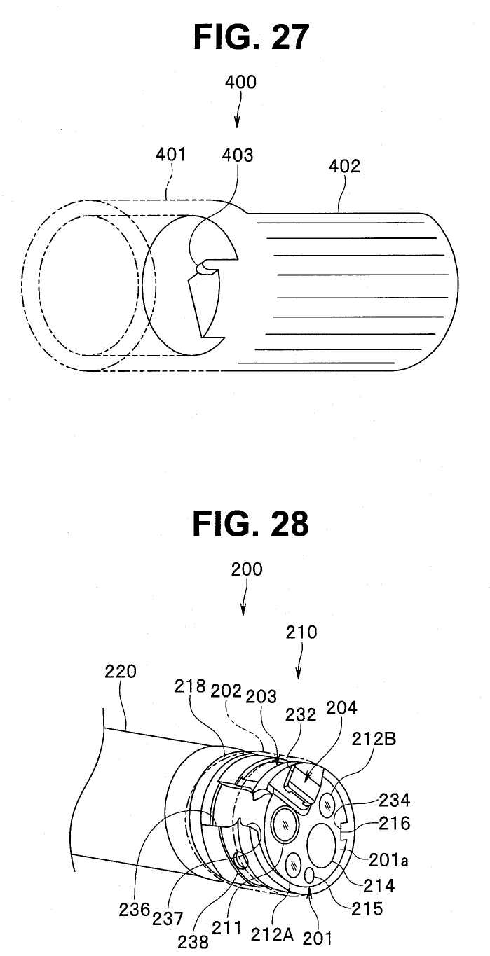

Next, a procedure for detaching the first distal end cover 202 and the second distal end cover 203 from the distal end member 201 (hereinafter referred to as procedure for detaching) will be described. A detaching jig 400 illustrated in FIG. 27 is used in the procedure for detaching. The detaching jig 400 includes a cylindrical portion 401 having a cylindrical shape with one end opened, a grasping portion 402 coupled to the cylindrical portion 401, and a claw portion 403 that is a protrusion formed inside the cylindrical portion 401. The cylindrical portion 401 is depicted by a dashed-and-double-dotted line in FIG. 27. The inner diameter of the cylindrical portion 401 is larger than the outer diameter of the second distal end cover 203. The claw portion 403 is configured to engage with the notch portion 237 of the second distal end cover 203.

Hereinafter, the procedure for detaching will be described with reference to FIG. 23, FIG. 24, and FIG. 27. In the procedure for detaching, firstly, the distal end portion 210 is covered by the cylindrical portion 401 of the detaching jig 400 such that the claw portion 403 of the detachment jig 400 engages with the notch portion 237 of the second distal end cover 203. Next, the detaching jig 400 is rotated in the direction around the insertion axis X of the insertion portion 200 in a state where the claw portion 403 is engaged with the notch portion 237. The rotation direction of the detaching jig 400 is preferably a counterclockwise direction, when the end surface 201a of the distal end member 201 is viewed in a direction parallel to the insertion axis X of the insertion portion 200 from the distal end side of the insertion portion 200. With this, the portion between the notch portion 236 and the notch portion 237 in the second distal end cover 203, that is, the fragile portion 235 is broken. FIG. 28 illustrates a state in which the fragile portion 235 is broken.

When the fragile portion 235 is broken, the engagement hole 238 of the second distal end cover 203 is disengaged from the locking pin 217 of the distal end member 201. In the procedure for detaching, then, the detaching jig 400 is removed from the distal end portion 210. The first distal end cover 202 and the second distal end cover 203 in which the fragile portion 235 is broken are pulled out from the distal end member 201. Thus, the first distal end cover 202 and the second distal end cover 203 are detached from the distal end member 201.

It is preferable that the arrangement of the nozzle configuring portion 232, the restricting protrusion 234, and the notch portion 237 in the second distal end cover 203 be determined based on the above-described rotation direction of the detaching jig 400. Specifically, in the mounted state, it is preferable that the nozzle configuring portion 232 be positioned in the rear side of the rotation direction relative to the notch portion 237, and the restricting protrusion 234 be positioned in the front side of the rotation direction relative to the notch portion 237 when the second distal end cover 203 is viewed in the direction parallel to the insertion axis X of the insertion portion 200 from the distal end side of the insertion portion 200. With this, an unnecessary external force may be prevented to apply to the fluid passage 213 of the distal end member 201 via the nozzle configuring portion 232 upon breaking the fragile portion 235, and as a result, the deformation or the breakage of such as the opening portion of the fluid passage 213 may be prevented. In addition, it is possible to prevent fragments of the second distal end cover 203, which are generated upon breaking the fragile portion 235, from entering the fluid passage 213.

The present invention is not limited to the embodiments described above, and various changes and modifications can be made without departing from the spirit and scope of the present invention.

What is claimed is:

1. An endoscope, comprising:
a distal end member provided at a distal end portion of an insertion portion configured to be inserted in a subject and including a fluid passage for allowing a fluid to flow;
a first distal end cover made of an elastically deformable material and detachably attached to the distal end member;
a second distal end cover made of a material harder than the material of the first distal end cover and attached to the distal end member;
a nozzle including a nozzle channel portion communicating with the fluid passage in a mounted state in which the first distal end cover and the second distal end cover are attached to the distal end member, and an ejection port positioned at an end of the nozzle channel portion and configured to eject the fluid to an outside; and
a first nozzle configuring portion provided to the first distal end cover, configuring at least part of the nozzle, and configuring at least part of a bending portion having a bent shape in the nozzle channel portion, wherein
the first nozzle configuring portion is covered by the second distal end cover in the mounted state.

2. The endoscope according to claim 1, wherein
at least part of the ejection port is configured by the first nozzle configuring portion.

3. The endoscope according to claim 2, further comprising:
a second nozzle configuring portion provided to the distal end member and configuring another part of the nozzle, wherein
the ejection port is configured by the first nozzle configuring portion and the second nozzle configuring portion.

4. The endoscope according to claim 3, wherein
the nozzle channel portion is configured by the first nozzle configuring portion and the second nozzle configuring portion.

5. The endoscope according to claim 3, wherein
the first nozzle configuring portion is sandwiched between the second distal end cover and the second nozzle configuring portion, and the position of the first nozzle configuring portion is restricted in the mounted state.

6. The endoscope according to claim 5, wherein
at least part of the first nozzle configuring portion is positioned between the second distal end cover and the second nozzle configuring portion in a cross-section perpendicular to an insertion axis of the insertion portion in the mounted state.

7. The endoscope according to claim 1, wherein
the first nozzle configuring portion configures the entire nozzle.

8. The endoscope according to claim 7, wherein
the first nozzle configuring portion is sandwiched between the second distal end cover and the distal end member, and the position of the first nozzle configuring portion is restricted in the mounted state.

9. The endoscope according to claim 8, wherein
at least part of the first nozzle configuring portion is positioned between the second distal end cover and the distal end member in a cross-section perpendicular to an insertion axis of the insertion portion in the mounted state.

10. The endoscope according to claim 1, wherein
the second distal end cover includes a breakable fragile portion when the second distal end cover is detached from the distal end member.

11. The endoscope according to claim 1, wherein
part of the first distal end cover is exposed in the mounted state.

12. The endoscope according to claim 11, wherein
part of the first nozzle configuring portion is exposed in the mounted state.

13. A distal end cover detachably attached to a distal end member including a fluid passage for allowing a fluid to flow through at a distal end portion of an insertion portion of an endoscope, the distal end cover comprising:
a first cover made of an elastically deformable material and detachably attached to the distal end member;
a second cover made of a material harder than the material of the first cover and attached to the distal end member;
a nozzle including a nozzle channel portion communicating with the fluid passage in a mounted state in which the first cover and the second cover are attached to the distal end member, and an ejection port positioned at an end of the nozzle channel portion and configured to eject the fluid to an outside; and
a first nozzle configuring portion provided to the first cover, configuring at least part of the nozzle, and configuring at least part of a bending portion having a bent shape in the nozzle channel portion, wherein
the first nozzle configuring portion is covered by the second cover in the mounted state.
